(19) United States
(12) Patent Application Publication
(10) Pub. No.: US 2005/0106646 A1
(43) Pub. Date: May 19, 2005

(54) In vitro serological diagnosis method of diagnosing infective endocarditis

(76) Inventor: Didier Raoult, Marseille (FR)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/507,646

(22) PCT Filed: Mar. 11, 2003
(86) PCT No.: PCT/FR03/00783

(30) Foreign Application Priority Data
Mar. 12, 2002 (FR) 02/03050

(51) Int. Cl. G01N 33/554; G01N 33/569

(52) U.S. Cl. 435/7.32; 530/388.4

(57) ABSTRACT

The present invention pertains to an in vitro serological diagnosis method for blood culture-negative infective endocarditis caused by the bacteria Coxellia burnetii and Bartonella sp in a patient suffering from a heart valve disorder, wherein an assay is performed of type IgG antibodies of the bacterium Coxellia burnetii phase 1 and of type IgG antibodies of a Bartonella sp bacterium, on one same serum specimen from the said patient, and it is determined whether the titre of said IgG antibodies is equal to or greater than 1:800.

The present invention pertains to an in vitro serological diagnosis method for blood culture-negative infective endocarditis in patients suffering from an endocarditis, and more particularly an infection by one of the two most frequent bacteria in blood culture-negative endocarditis, namely Coxellia burnetii and Bartonella sp.

The present invention also concerns a diagnosis kit for implementing said in vitro serological diagnosis method.

Bacteria of the genus Bartonella sp and Coxiella burnetii are involved in a large number of diseases. The different forms of the diseases connected with the Bartonella bacterium are the following: cat scratch disease, bacillary angiomatosis, trench fever, peliosis hepatitis and endocarditis.

The diseases due to infections caused by a bacterium of Coxiella type are defined as Q fever and may translate as:
acute infections: fever, hepatitis, pneumonia, myocarditis, pericarditis or meningitis,
chronic infections: endocarditis, pericarditis, vasculitis, osteitis, lung tumour or repeated abortion.

Endocardites arise from bacterial infection of a heart valve. Patients suffering from endocarditis therefore have a valve lesion identifiable when taking the patient's history (illness known to the patient) by medical examination (heart murmur) or X-ray examination (echocardiography). The diagnosis of endocarditis caused by ordinary bacteria is based on blood culture. There are culture-negative endocardites whose cause may be (but not necessarily) an infection with Bartonella or Coxiella bacteria.

Also, some carriers of acute Q fever or an infection do not suffer from endocarditis.

At the present time, when it is wished to diagnose a blood culture-negative endocarditis, in particular an endocarditis related to infection with Coxiella burnetii or a bacterium of Bartonella type, recourse needs to be made to specific serology for each of these two types of bacteria [(Raoult D. et al., Ann. Intern. Med., 1996, 125 :646-652 and Drancourt M., et al., New Engl J Med. 1995, 332 :419-423) and the bacterium Bartonella quintana deposited with the ATCC collection under number 49193].

Currently, the diagnosis of diseases related to an infection with the bacterium Coxiella burnetii is made by parallel assay of antibodies against phase 1 antigens and of phase II antibodies which are the two different specific forms of the bacterium Coxiella burnetii. Endocardites are usually accompanied by a substantial increase in antibodies directed against phase 1 at the same time as those directed against phase II. The serum to be tested is screened separately for the two forms, phase 1 and phase II of the bacterium Coxiella burnetii, using different diagnosis kits. Also, this screening is made by detecting all immunoglobulins (IgG, IgM and IgA) or by separate testing of the immunoglobulins G, M and A. Serology may e indicated for acute infections, namely fever, hepatitis, pneumonia, myocarditis, pericarditis, meningitis, or chronic infections, namely endocarditis, pericarditis, vasculitis, osteitis, lung tumour, repeated abortion. When results are positive, a quantitative evaluation is made of the antibodies by means of test involving increasing dilutions of each type of immunoglobulin at dilutions ranging form 1:50 to 1:3200 in order to determine the exact titre.

Also, at the present time, the diagnosis methods for bacterial diseases with bacteria of Bartonella type are performed with antigens which are either the bacterium Bartonella henselae or Bartonella quintana which are tested at successive dilutions detecting the presence of total immunoglobulin or in some cases IgGM. In this case, the same serum testing is prescribed for the different forms of infection with Bartonella, namely cat scratch disease, bacillary angiomatosis, bacteraemia, trench fever, peliosis hepatitis and endocarditis.

It is known that approximately 80% of blood culture-negative endocardites in France are caused by these bacteria (Coxiella burnetii accounting for around 50% and Bartonella bacteria for around 30%)

The treatments for endocardites are evidently very different according to the infection under consideration.

At the present time there is no diagnosis kit available for the specific serological testing of endocardites, in particular to detect endocardites specific to bacterial infections with Coxiella burnetii or Bartonella.

The object of the present invention is to provide an in vitro diagnosis method that is easy to use, quick, requiring only one test to determine the presence or not of an endocarditis through Bartonella sp or Coxiella burnetii in patients suffering from a heart valve disorder.

For this purpose, the present invention provides an in vitro serological diagnosis method for blood culture-negative infective endocardites caused by the bacteria Coxellia burnetii and Bartonella sp in a patient suffering from a heart valve disorder, characterized in that an assay is performed of IgG type antibodies directed against the bacterium Coxellia burnetii phase I and of IgG type antibodies directed against the bacterium Bartonella sp, on one same serum specimen from said patient, and it is determined whether the titre of said IgG antibodies is 1:800 or more, i.e. it is verified whether it is possible to detect the presence of said IgG antibodies in a patient serum specimen diluted to 1:800.

The present invention provides a quick, simple test by testing a single dilution of a serum specimen, making it not only possible to ascertain that the patient carries these antibodies, but also to ascertain that there is a given high level of antibodies to enable identification of said endocarditis. More particularly, with the present invention it is possible on one single serum specimen, especially using an indirect immunofluorescence technique with single dilution, to detect an infection with one of the two most frequent bacteria in culture-negative endocarditis, namely Coxellia burnetii and Bartonella sp, in a patient suffering from a heart valve disorder found at clinical or echocardiography examination. With the present invention it was possible to determine a single concentration of anti-IgG antibodies of the bacterium Coxellia burnetii phase 1 and of a Bartonella sp bacterium from which, in a specimen from a patient suffering from endocarditis, results can conclude the presence of endocarditis with one of these bacteria.

In one particular, advantageous embodiment, an assay is performed in which the two said bacteria-antigens are deposited on a solid support, and a single dilution of the serum to be tested is made to which is added an excess anti-IgG immunoglobulin, preferably labelled, which is caused to react with said solid support.

According to the present invention, solely the Coxiella burnetii phase 1 bacterium is used, since it was observed that a high level of antibodies against phase 1, in particular over and above 1:800, only occurs in the chronic forms of infection with Coxiella burneti including endocardites. Also, these antibody levels of over 1:800 are specific to a chronic infection belonging to a bacterium of the Bartonella genus, including endocardites.

Consequently, for patients suffering from an endocardiac disorder, the presence of antibodies against one of these two antigens at a level of over 1:800 is necessarily related to an infection with one of these two bacteria, since the only chronic disorder connected with these two bacteria which causes heart disorders is of endocarditis type.

In addition, according to the present invention, it was observed that the detection of antibodies between the bacterium Coxiella burnetii or the bacterium Bartonella of IgM type could be due to the presence of a rheumatoid factor. In most cases rheumatoid factors are immunoglobulins of IgM anti-IgG type, and are likely to give false positive results when IgG levels are low in patients suffering from endocarditis caused by other bacteria, whereas patients suffering from endocarditis often have rheumatoid factors. This is why, according to the present invention, specific detection is made of antibodies directed against IgGs (and not IgMs or total immunoglobulins) so as to eliminate false positives due to low IgG levels associated with rheumatoid factors (which are IgM anti-IgG ).

The test of the present invention represents simplification and considerable time saving compared with currently used techniques for screening and diagnosis. No screening dilution is tested and only one test per serum specimen is performed. A dilution of 1:800 was determined which represents the best sensitivity-specificity ratio applied to the two bacteria, so that it is possible to group together the detection of responsible bacteria in one single test. According to the present invention, an original syndrome approach is used for diagnosis and not a bacterial disease approach as previously, by limiting the question raised serologically to the problem of the diagnosis of endocarditis and by replacing all the serum techniques subsequently applied with a single technique which achieves screening and diagnosis on a single titre.

In one advantageous embodiment, an assay is performed in which:
1. the two said bacteria are placed on a solid support, and
2. said bacteria are caused to react with a serum specimen of said patient, diluted to 1:800 to which a human anti-IgG immunoglobulin has been added, preferably labelled, and
3. it is verified whether said human anti-IgG immunoglobulins, preferably labelled, bind to said solid support.

In other words, if after washing said support it is found that said labelled anti-IgG immunoglobulins are bound to the support, their binding to said solid support can only have occurred via IgG antibodies (Ac) directed against said bacterium (Ag) forming an Ag-Ac-Ig anti IgG complex.

In one preferred embodiment, said bacterium is Bartonella henselae or Bartonella quintana.

In one particular embodiment, the following bacteria are placed on said solid support:
a phase 1 Coxellia burnetii bacterium,
a Bartonella quintana bacterium.

Phase 1 Coxellia burnetii and Bartonella bacteria are accessible to the public through various sources. They have been described in the literature [Tissot Dupont H., et al. Clin. Diag. Lab. Immunol., 1994, 1 :189-196], and several have been deposited with the ATCC collection, in particular the phase 1 Coxellia burnetii bacterium deposited with the ATCC under number VR615.

As solid support, any device may be used that is suitable for handling cell and bacterial suspensions, in particular tubes, glass slides, Bijoux tubes or rigid microtiter plates in polyethylene, polystyrene, polyvinyl chloride or nitrocellulose containing microwells, glass slides being preferred.

For anti-human immunoglobulin labelling, the type advantageously used is enzymatic, radioactive or fluorescent labelling, the latter type being preferred.

The expression <<fluorescent labelling>> means that the antibody was made fluorescent by coupling to or complexing with a suitable fluorescent agent such as fluorescein isothiocyanate.

By <<radioactive labelling>> is meant that the antibody carries a radioactive isotope enabling its assay by the counting of associated radioactivity, the isotope possibly being carried either on a structural element of the antibody, for example constituent tyrosine residues, or on a suitable radical fixed to it.

By <<enzymatic labelling>> is meant that the specific antibody is coupled to or complexed with an enzyme which, associated with the use of suitable reagents, enables quantitative measurement of this specific antibody.

The substrate and reagents are chosen so that the end reaction or reaction sequence product caused by the enzyme and using these substances is:
either a coloured or fluorescent substance which diffuses in the liquid medium surrounding the tested sample and which is either subjected to final spectrophotometric or fluorimetric measurement respectively, or is visually assessed; optionally with respect to a range of calibrated shades,
or an insoluble coloured substance which is deposited on the tested sample and which can either be measured by reflection photometry or assessed visually optionally with respect to a range of calibrated shades.

When an immunoglobulin which has been made fluorescent is used, the fluorescence associated with the tested sample is directly read on a suitable apparatus.

When a radioactive probe is used, such as iodine 125 for example, the radioactivity associated with the tested sample is counted in a gamma counter using an appropriate modality for example after solubilising the cells with an alkaline solution (a sodium hydroxide solution for example) and recovery of the solution containing the radioactivity using an absorbent buffer.

When an enzyme on the specific antibody is used, the onset of a coloured or fluorescent product is obtained by adding a solution containing the substrate of the enzyme and one or more ancillary reagents so that finally a reaction product is obtained that is either a coloured product soluble in the medium, or an insoluble coloured product, or a soluble fluorescent product as explained above. The light signal from the samples so treated is then measured using apparatus adapted to each case: transmission photometer, or reflection photometer or fluorimeter respectively. Alternatively, it is also possible to assess the colouring obtained visually, optionally with the help of a range of calibrated coloured solutions.

If the enzyme used is alkaline phosphatase, the coupling of this enzyme with the specific antibody is conducted following the method described by Boehringer Mannheim-Biochemica. The preferred substrates of this enzyme are paranitrophenylphosphate for fluorometric reading, or bromo-5 chloro-4-umbelliferyl phosphate for fluorometric reading, or bromo-5 chloro-4 indolyl-6 phosphate to obtain an insoluble coloured reaction product. It is also possible to use β-galactosidase as enzyme whose preferred substrates are orthonitrophenyl β-D-galactropyranoside or methyl-4 umbelliferyl β-D-galactopyranoside.

Preferably, the specific antibodies can be coupled to peroxidase. In this case, the coupling method is derived from the method described by M. B. Wilson and P. K. Nakane in: Immunofluorescence and related staining techniques, W. Knapp, K. Kolubar, G. Wicks ed. Elsevier/North Holland. Amsterdam 1978, p. 215-224.

The reagents used to develop the peroxidase conjugated with the specific antibodies contain hydrogen peroxide, a substrate of the enzyme, and a suitable chromogen for example orthophenylenediamine or azino-2-2′ bis (3-ethylbenzthiazoline-6-sulphonic acid), or ABTS, to obtain a coloured end reaction product soluble in the medium, or further diamino-3,3′ benzidine or amino-3 ethyl-9 carbazole or chloro-4 α-naphtol to obtain an insoluble end reaction product, or further proprionic parahydroxyphenyl acid to obtain a fluorescent reaction product soluble in the medium.

A further embodiment of the invention is the use of immunoglobulins coupled to acetylcholinesterase.

The acetylcholinesterase is coupled to the antibody preferably following a method derived from the method described in French patent n^{o} 2 550 799 or a method which schematically entails the preparing of antibody fragments using a known technique, modifying the enzyme by reaction with a suitable heterobifunctional agent, and finally coupling of the products obtained. Other known methods for constructing immunoenzymatic conjugates may also be used in this case.

Development of the enzymatic activity specifically related to the antigen recognized by the acetylcholinesterase conjugate is preferably conducted with the well known technique using acetylthiocholine as the enzyme substrate and Ellman's reagent, or dithio-5,5′ nitro-2 benzoic acid as chromogen, using any variant adapted to the case being examined, for example the variant described by Pradelles et al., in Anal. Chem. 1985, 57 :1170-1173.

The cited chromogens are used as such or in the form of water-soluble salts.

Other characteristics and advantages of the present invention will become apparent in the light of the following description, describing the detailed experiment to carry out the invention, which are given solely for illustration purposes.

1-The Antigens

Bartonella quintana [strain: Oklahoma (ATCC N^{o} 49193) and strain: Marseille (Faculté de Médecine, 27 Boulevard Jean Moulin 13385 Marseille cedex 5—Rickettsiae Unit collection)] Bartonella henselae [strain Houston (ATCC N^{o} 49882) and strain: Marseille (Faculté de Médecine , 27 Boulevard Jean Moulin 13385 Marseille cedex 5—Rickettsiae Unit collection)] were used as antigens for the Bartonella bacteria. The antigen of Bartonella quintana is obtained as follows: the bacterium is cultured in gelose with 10% fresh blood. The strain used was Oklahoma. This strain was used to inoculate cultured cells which were human endothelial cells, in a cell line called ECV. 24 Hours after inoculation these cells were harvested. The antigens were semi-purified, consisting of removing cell debris by centrifuging, then fixing with sodium azide before use.

The bacteria were cultured on an endothelial cell line (ECV 304), harvested, adjusted to 1 mg/ml and directly used as antigen. This antigen production has been described in the literature [D. Raoult., H, Tissot-Dupont., M. Enea Mutillod., Clin. Infect. Dis., 1994, 19 :335] [Raoult D., Fournier P E., Drancourt M. et al, Ann. Intern. Med., 1996, 125 :646-652].

The antigens of Coxiella burnetii were produced using the Nine Mile strain (ATCC N^{o} VR615). To obtain the antigen of phase 1, the strain was injected into a BalbC mouse via intraperitoneal route. On day 7, the spleen of this mouse was harvested, ground and inoculated into these Vero cells in culture. Once the bacterium had proliferated within the Vero cells, the bacteria were harvested as already described in the literature and were used to re-inoculate other cells. The phase 1 antigen was obtained in the first 6 culture passes on Vero cells. Phase II antigens were obtained on and after the 15^{th} pass on Vero cells. Phase II is a mutant obtained by phase I culture. The antigen of Coxiella burnetii was harvested and subjected to two successive centrifugations to obtain a semi-purified antigen, adjusted to 1 mg/ml. This technique has been described [Fournier P E., Marrie T J., Raoult D. J. Clin. Microbiol., 1998, 36 :1823-1834]. These antigens were spotted onto a Dynatech® ten-hole Teflon-coated slide.

2-The Tested Sera

To develop the technique, the inventor selected a certain number of serum specimens from patients with identified pathology. These were patients with Q fever or Bartonella endocardites, and patients with endocarditis caused by other bacteria in order to obtain negative controls, but also patients with acute Q fever (with no endocarditis), patients carrying cat scratch disease (infection caused by Bartonella but with no endocarditis) and also patients suffering from other identified diseases (Cytomegalovirus infection, Epstein Barr virus infection).

244 serum specimens were tested. The selected sera consisted of 40 specimens from patients donating blood suffering from no disease, 15 sera from patients suffering from cat scratch disease diagnosed by other means (PCR on gland), 15 patients who had suffered from acute Q fever (diagnosed by evidencing of seroconversion), 30 patients diagnosed with Q fever endocarditis (diagnosed by detection of the bacterium on the patient's heart valves) and sera from 20 patients presenting with Bartonella endocarditis (diagnosed by culture or PCR of patient's heart valves). Also, 99 sera from patients with endocarditis whose aetiology was not Coxiella burnetii or Bartonella were tested as well as a panel of patients with documented infection from another disease (neither endocarditis nor infection with Bartonella or Coxiella). In addition, 25 patients suffering from another disease were tested. These were 5 patients with infectious mononucleosis, 5 patients with Cytomegalovirus infection, 5 patients with herpes virus seroconversion, 5 patients with hepatitis B and 5 patients with HIV virus seroconversion, the AIDS agent. Among the patients with endocarditis, 11 presented with a rheumatoid factor detected by the latex test (Rapitex, R F. Dade Behring))

3-Serological Technique

The serological technique consists of depositing the 6 antigens on a slide [Bartonella quintana Marseille, Bartonella quintana Oklahoma, Bartonella henselae Houston (ATCC N^{o} 49882)] and Marseille and Coxiella burnetii phase I and phase II (Faculté de Médecine, 27 Boulevard Jean Moulin 13385 Marseille cedex 5—Rickettsiae Unit collection).

The slides were glass slides with 20 spots. These slides were cleaned, the antigen spotted or micropipetted onto the surface tangentially to the upper part of the circle for Coxiella burnetii, then with another pipette for Bartonella quintana to the lower part of the same spot on each spot. After depositing, the antigen was fixed by immersing the slide for ten minutes in acetone. When the slide was dry the serum dilution was deposited.

Serum dilutions of 1:400, 1:800, 1:1600 and 1:3200 were tested in PBS. The sera diluted in PBS were deposited [D. Raoult., H, Tissot-Dupont., M. Enea Mutillod., Clin. Infect. Dis., 1994, 19 :335], left to incubate for 30 minutes, washed three times. The human anti-immunoglobulin antibody was then added at the manufacturer's recommended dilution, namely 1:400. The antibody used (Bio Mérieux) was either a total anti-immunoglobulin antibody (against a mixture of IgG, IgM, IgA) or an antibody specifically against the IgGs. The slides were incubated for thirty minutes then washed three times ten minutes in PBS and dried. A glass coverslip was placed on the slide with a drop of glycerin between the slide and coverslip and observed under immunofluorescence. Each slide comprised a positive control and a negative control, and the spots were read under fluorescence microscopy with 400 magnification.

4-Results

The occurrence of a definite reaction against either one of the antigens indicates that the endocarditis diagnosed in the patient sampled, is an endocarditis with the bacterium showing the positive reaction. The presence of the two antigens is warranted by the fact that cross-reactions exist between the two, but they generally concern infections with Coxiella burnetii having low-titre Bartonella antibodies. If both reactions are positive this may require a more complete test with Coxiella burnetii. Finally these are the two bacteria which account for the major part of culture-negative endocardites. In a recent series by the Ricketssiae unit [Houpikian P. Diagnostic des endocardites a hémocultures négatives. These de Médecine, Marseille, 2001.], 50% of 300 culture-negative endocardites were caused by Coxiella burnetii and 30% by Bartonella.

The results show that the detection of total immunoglobulin raised a problem of specificity. Two patients suffering from endocarditis caused by banal bacteria and carrying rheumatoid factors showed antibodies of 1:800 against Bartonella. Secondly, this work showed that it is not necessary to test both phase I and phase II Coxiella burnetii since all endocardites had antibodies against phase I of 1:800 or over, whereas only one Q fever without endocarditis had antibodies against phase I of 1:800 or over. On the other hand, while all the endocardites also had antibodies against phase II at a titre of 1:800 or over, 10 of the Q fevers had antibodies against phase II of 1:800 or over. It was therefore decided no longer to test phase II and only to use phase I for the diagnosis of endocardites.

Regarding the Bartonella infections, irrespective of the cause of the Bartonella endocarditis (Bartonella henselae, Bartonella quintana) the antigen of Bartonella quintana Marseille or Oklahoma allowed the serum dilution of 1:800 to identify all endocarditis causes. There were 15 endocardites with Bartonella quintana and 5 endocardites with Bartonella henselae and all were detected by the antigens of Bartonella quintana.

A dilution of 1:1600 only detected 11 out of 20 endocardites with Bartonella. On the other hand, a dilution of 1:800 enabled detection of all Bartonella. endocardites. All Coxiella burnetii endocardites were also detected at 1:800 and 1:1600. At a dilution of 1:800 none of the negative controls were positive for Bartonella or Coxiella burnetii, among those patients suffering either from cat scratch disease or endocarditis caused by other bacteria or from other diseases. Overall, the 244 sera tested included 50 endocardites with Coxiella burnetii or Bartonella henselae or B. quintana. Immunofluorescence serology using only Bartonella quintana and Coxiella burnetii phase 1 antigens gave 50 positive results for the 50 endocardites and 1 positive result for an acute Q fever.

If the value of the serology is assessed on endocarditis cases among 149 cases tested, no endocarditis caused by a bacterium other Bartonella or Coxiella burnetii was detected with this technique only using IgGs against Coxiella burnetii phase I and Bartonella quintana.

1. In vitro serological diagnosis method for blood culture-negative infective endocarditis caused by the bacteria Coxellia burnetii and Bartonella sp in a patient suffering from heart valve disorder, wherein an assay is performed of type IgG antibodies directed against Coxellia burnetii phase I bacterium and of type IgG antibodies directed against the bacterium Bartonella sp, on one same serum specimen from said patient, and it is determined whether the titre of said IgG antibodies is equal to or greater than 1:800.

2. Diagnosis method as in claim 1, wherein an assay is performed in which:
(a) the two said bacteria are deposited on a solid (support, and
(b) said bacteria are caused to react with a serum of said patient diluted to 1:800, to which has been added a human anti-IgG immunoglobullin, preferably labelled, and
(c) it is verified whether said human anti-IgG immunoglobulins, preferably labelled, bind to said solid support.

3. Diagnosis method as in claim 1, wherein said Bartonella sp bacterium is a Bartonella quintana or Bartonella henselae bacterium.

4. Diagnosis method as in claim 1, wherein the following bacteria are deposited on said solid support:
a phase 1 Coxellia burnetii bacterium, and
a Bartonella quintana bacterium.

5. Diagnosis method as in claim 1, wherein the human anti-IgG immunoglobulin comprises labelling, preferably detectable by fluorescence.

6. Diagnosis kit for use with a diagnosis method as claim 1, wherein it contains:
a solid support on which said bacteria have been deposited, and
a solution comprising a human anti-IgG immunoglobulin, preferably labelled.

7. Diagnosis kit for use with a diagnosis method as claim 2, wherein it contains:
a solid support on which said bacteria have been deposited, and
a solution comprising a human anti-IgG immunoglobulin, preferably labelled.

8. Diagnosis kit for use with a diagnosis method as claim 3, wherein it contains:
a solid support on which said bacteria have been deposited, and
a solution comprising a human anti-IgG immunoglobulin, preferably labelled.

9. Diagnosis kit for use with a diagnosis method as claim 4, wherein it contains:
a solid support on which said bacteria have been deposited, and
a solution comprising a human anti-IgG immunoglobulin, preferably labelled.

10. Diagnosis kit for use with a diagnosis method as claim 5, wherein it contains:
a solid support on which said bacteria have been deposited, and
a solution comprising a human anti-IgG immunoglobulin, preferably labelled.
